(19) United States
(12) Patent Application Publication
(10) Pub. No.: US 2023/0215564 A1
(43) Pub. Date: Jul. 6, 2023

(54) DIGITAL DOCUMENT ASSISTANT FOR ORGANIZING MEDICAL INFORMATION

(71) Applicant: International Business Machines Corporation, Armonk, NY (US)

(72) Inventors: Neela Vauhini Komaragiri, Visakhapatnam (IN); Saraswathi Sailaja Perumalla, Hyderabad (IN); Raghupatruni Nagesh, Hyderabad (IN); Omkar Nath Bag, Balangir (IN)

(21) Appl. No.: 17/647,025

(22) Filed: Jan. 5, 2022

(51) Int. Cl. G16H 50/20; G16H 50/70; G16H 10/60; G06F 16/93

(52) U.S. Cl. CPC G16H 50/20; G16H 50/70; G16H 10/60; G06F 16/93

(57) ABSTRACT

A document management system that stores all reports, bills, receipts, prescriptions, etc. by converting these pieces of data into a digital format. Once this data has been converted into a digital format, there is a method and system that is structured and configured to compartmentalize and classify the data using a digital content analysis module. Once this data is compartmentalized and classified, a user can conveniently access these disparate pieces of data in one place and make decisions that benefit the owner of the data.

### BACKGROUND

The present invention generally relates to the field of data analysis, and more specifically to utilizing enterprise-level methods for analyzing and maintaining the medical data of patients on a large-scale.

### SUMMARY

According to an aspect of the present invention, there is a method, computer program product and/or system that performs the following operations (not necessarily in the following order): (i) receiving a plurality of medical documents, with the plurality of medical documents including information indicative of a complete medical history of a user; (ii) responsive to the receipt of the plurality of medical documents, organizing information included in the complete medical history by category, with the categories including treatment(s) received, past ailment(s), and present ailment(s); (iii) creating a digital document repository to store the complete medical history, with the digital repository being accessible by the user and a set of authorized medical professionals; and (iv) storing the complete medical history in the digital document repository.

According to an aspect of the present invention, there is a method, computer program product and/or system that performs the following operations (not necessarily in the following order): (i) receiving a plurality of documents, with the plurality of documents including information indicative of a patient's medical history; (ii) determining, by a Natural Language Processing (NLP) module, the substantive content of the patient's medical history and the context for the patient's medical history; (iii) analyzing, by a digital documents analysis module, the substantive content of the patient's medical history; and (iv) responsive to the analysis, generating, by an insights orchestration module, medical insights for the patient with respect to a current medical condition.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a block diagram showing a machine logic (for example, software) portion of the first embodiment system;

FIG. 4 is a block diagram showing information that is helpful in understanding embodiments of the present invention;

FIG. 5 is a first architecture diagram showing information that is helpful in understanding embodiments of the present invention;

FIG. 6 is a second architecture diagram showing information that is helpful in understanding embodiments of the present invention; and

FIG. 7 is a system overview diagram showing information that is helpful in understanding embodiments of the present invention.

### DETAILED DESCRIPTION

Some embodiments of the present invention are directed towards a document management system that stores all reports, bills, receipts, prescriptions, etc. by converting these pieces of data into a digital format. Once this data has been converted into a digital format, there is a method and system that is structured and configured to compartmentalize and classify the data using a digital content analysis module. Once this data is compartmentalized and classified, a user can conveniently access these disparate pieces of data in one place and make decisions that benefit the owner of the data.

This Detailed Description section is divided into the following sub-sections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

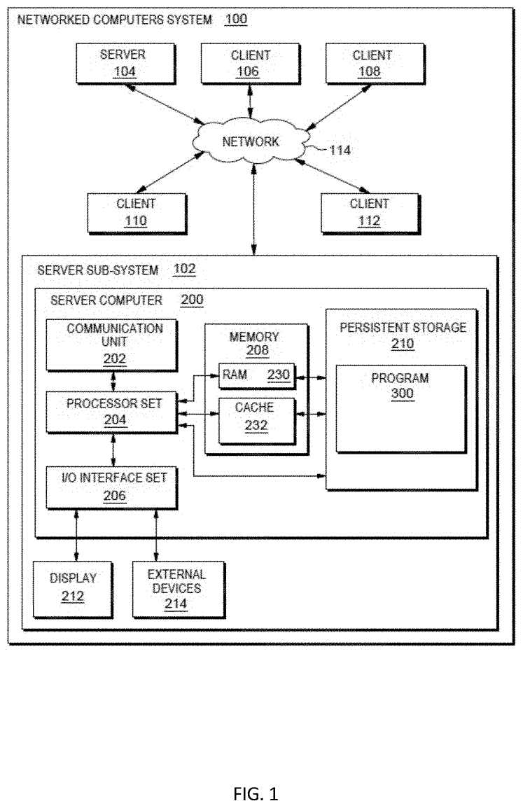

An embodiment of a possible hardware and software environment for software and/or methods according to the present invention will now be described in detail with reference to the Figures. FIG. 1 is a functional block diagram illustrating various portions of networked computers system 100, including: server sub-system 102; client sub-systems 104, 106, 108, 110, 112; communication network 114; server computer 200; communication unit 202; processor set 204; input/output (I/O) interface set 206; memory device 208; persistent storage device 210; display device 212; external device set 214; random access memory (RAM) devices 230; cache memory device 232; and program 300.

Sub-system 102 is, in many respects, representative of the various computer sub-system(s) in the present invention. Accordingly, several portions of sub-system 102 will now be discussed in the following paragraphs.

Sub-system 102 may be a laptop computer, tablet computer, netbook computer, personal computer (PC), a desktop computer, a personal digital assistant (PDA), a smart phone, or any programmable electronic device capable of communicating with the client sub-systems via network 114. Program 300 is a collection of machine readable instructions and/or data that is used to create, manage and control certain software functions that will be discussed in detail, below, in the Example Embodiment sub-section of this Detailed Description section.

Sub-system 102 is capable of communicating with other computer sub-systems via network 114. Network 114 can be, for example, a local area network (LAN), a wide area network (WAN) such as the Internet, or a combination of the two, and can include wired, wireless, or fiber optic connections. In general, network 114 can be any combination of connections and protocols that will support communications between server and client sub-systems.

Sub-system 102 is shown as a block diagram with many double arrows. These double arrows (no separate reference numerals) represent a communications fabric, which provides communications between various components of sub-system 102. This communications fabric can be implemented with any architecture designed for passing data and/or control information between processors (such as microprocessors, communications and network processors, etc.), system memory, peripheral devices, and any other hardware components within a system. For example, the communications fabric can be implemented, at least in part, with one or more buses.

Memory 208 and persistent storage 210 are computer-readable storage media. In general, memory 208 can include any suitable volatile or non-volatile computer-readable storage media. It is further noted that, now and/or in the near future: (i) external device(s) 214 may be able to supply, some or all, memory for sub-system 102; and/or (ii) devices external to sub-system 102 may be able to provide memory for sub-system 102.

Program 300 is stored in persistent storage 210 for access and/or execution by one or more of the respective computer processors 204, usually through one or more memories of memory 208. Persistent storage 210: (i) is at least more persistent than a signal in transit; (ii) stores the program (including its soft logic and/or data), on a tangible medium (such as magnetic or optical domains); and (iii) is substantially less persistent than permanent storage. Alternatively, data storage may be more persistent and/or permanent than the type of storage provided by persistent storage 210.

Program 300 may include both machine readable and performable instructions and/or substantive data (that is, the type of data stored in a database). In this particular embodiment, persistent storage 210 includes a magnetic hard disk drive. To name some possible variations, persistent storage 210 may include a solid state hard drive, a semiconductor storage device, read-only memory (ROM), erasable programmable read-only memory (EPROM), flash memory, or any other computer-readable storage media that is capable of storing program instructions or digital information.

The media used by persistent storage 210 may also be removable. For example, a removable hard drive may be used for persistent storage 210. Other examples include optical and magnetic disks, thumb drives, and smart cards that are inserted into a drive for transfer onto another computer-readable storage medium that is also part of persistent storage 210.

Communications unit 202, in these examples, provides for communications with other data processing systems or devices external to sub-system 102. In these examples, communications unit 202 includes one or more network interface cards. Communications unit 202 may provide communications through the use of either or both physical and wireless communications links. Any software modules discussed herein may be downloaded to a persistent storage device (such as persistent storage device 210) through a communications unit (such as communications unit 202).

I/O interface set 206 allows for input and output of data with other devices that may be connected locally in data communication with server computer 200. For example, I/O interface set 206 provides a connection to external device set 214. External device set 214 will typically include devices such as a keyboard, keypad, a touch screen, and/or some other suitable input device. External device set 214 can also include portable computer-readable storage media such as, for example, thumb drives, portable optical or magnetic disks, and memory cards. Software and data used to practice embodiments of the present invention, for example, program 300, can be stored on such portable computer-readable storage media. In these embodiments the relevant software may (or may not) be loaded, in whole or in part, onto persistent storage device 210 via I/O interface set 206. I/O interface set 206 also connects in data communication with display device 212.

Display device 212 provides a mechanism to display data to a user and may be, for example, a computer monitor or a smart phone display screen.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

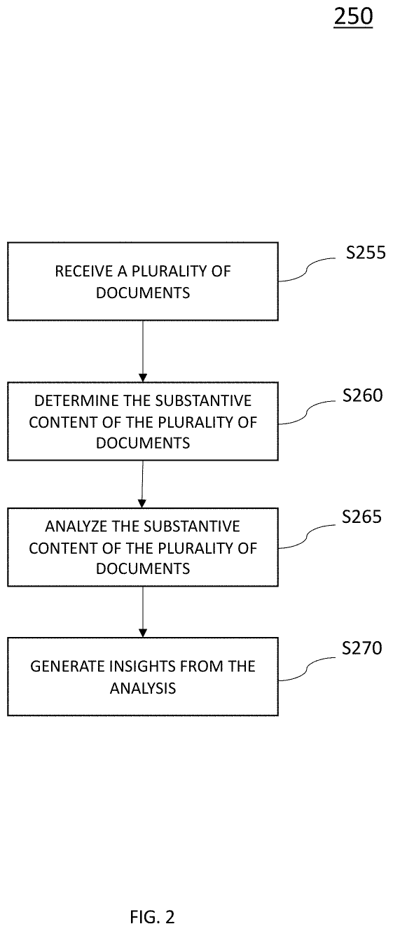

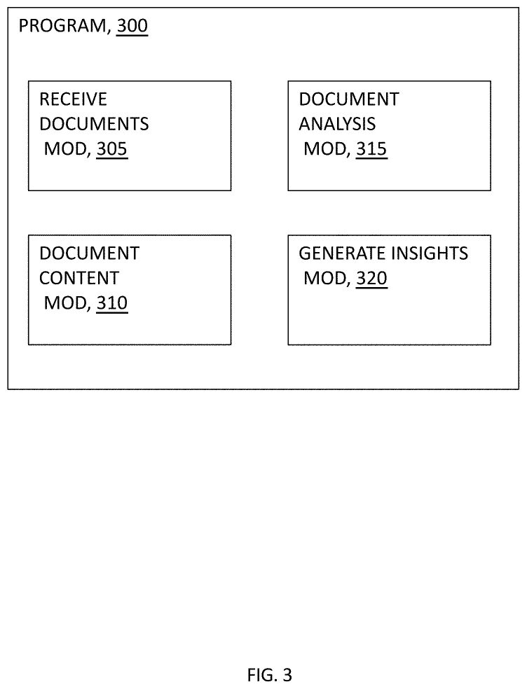

FIG. 2 shows flowchart 250 depicting a method according to the present invention. FIG. 3 shows program 300 for performing at least some of the method operations of flowchart 250. This method and associated software will now be discussed, over the course of the following paragraphs, with extensive reference to FIG. 2 (for the method operation blocks) and FIG. 3 (for the software blocks).

Processing begins at operation S255, where receive documents module (“mod”) 305 receives a plurality of medical documents. In this example, the documents are related to a single user or a single event. According to some embodiments of the present invention, the documents are medical records making up a complete medical history of the user. The medical records provide information relating to a patient's past treatments that he or she was recommended, past ailment(s), present ailment(s) (if there are any), and past treatments that the patient received.

Processing proceeds to operation S260, where organize data mod 310 organizes the information included in the patient's complete medical history (discussed in connection with receiving the plurality of medical documents in operation S255, above). The documents contained in the patient's medical history will contain a variety of relevant medical and non-medical information, such as treatments received (currently or previously), medical procedures that the patient has undergone, ailments that the patient may have experienced or is currently experiencing, and so on. These documents also contain information related to the expenses incurred by the patient for the various treatments received and the cost of his or her insurance premiums at the time of treatment.

In order to organize this information that is collectively contained in the plurality of medical documents, embodiments of the present invention use Natural Language Processing (NLP) and Artificial Intelligence (AI) techniques to extract and analyze the language that is contained in these documents in order to properly document and categorize the medical and non-medical information included therein.

Processing proceeds to operation S265, where create repository mod 315 creates a digital document repository. In some embodiments of the present invention, the digital document repository is a centralized server that is structured and configured to store and securely maintain the patient's medical documents. The digital document repository is accessible by the patient. Additionally, the patient can provide a set of authorization keys to other users, such as doctors, dentists, pharmacists, insurance providers, etc. so that these users can properly utilize the information stored in the digital document repository for the patient's benefit.

In some embodiments of the present invention, these authorization keys can be provided to the users by the patient on a permanent or temporary basis, with the patient having full authority to revoke access to the digital document repository at any time.

Processing finally proceeds to operation S270, where data storage mod 320 stores the patient's complete medical history in the digital document repository. Once the digital document repository is created and configured for use by the patient, the patient's complete medical history can be stored and accessed by all authorized parties in order to make proper medical decisions (such as treatment options) and non-medical decisions (such as financial considerations relating to any potential treatment that a patient may receive).

### III. Further Comments and/or Embodiments

Typically for any medical practitioner to review medical reports of a user, it is mandatory to understand the seriousness of a medical issue. In order for the patient to keep track of history of ailments and the medicines prescribed, it is a common practice to rely on hard copies of medical reports and medical prescriptions. It is relatively easy to misplace these reports or for the documents to get lost. For the sake of convenience and efficient use, embodiments of the present invention provide a repository for such important documents and it can be a “one stop” place for all of the confidential information of a given patient.

For specific medical problems, patients typically visit multiple doctors and secure multiple prescriptions. It can become a tedious task to store and safely maintain such history of events to be able to refer to them when a particular ailment appears (or reappears). In order to make it easy for the patient and the doctor, embodiments of the present invention create a data storage bank exclusively for these medical reports, analysis, results of tests and the prescribed list of medicines.

Consequently, whenever a given patient visits a medical professional, all the patient would need is a customer ID. This customer ID carries a history of ailments and the treatment done and can accordingly proceed with providing treatment recommendations. Embodiments of the present invention do not require the patient to describe their entire medical history when there is a high chance the it is not supported by specific dates, a sequence of events, or proper documentation. Additionally, this can be applied to various insurances, policies, or bonds that the patient has on his or her name. Essentially, it is a soft copy and can reduce the carbon footprint as well.

With respect to domestic digital documents:

In a given domestic organization, there will be various activities and tasks carried by each member and only a few transactions will have reference documents (for example, medical bills, medical reports, electricity bills, grocery purchase bills, educational certifications, vehicle insurance documents, etc.). Each document has a specific need and is sometimes needed in the future for financial planning purposes or medical treatment purposes. In domestic organizations where more members are contributing to the income, managing financial aspects of these documents can sometimes be complex. There is a need to manage all relevant bills and reports as digital documents that can be referred to whenever they are needed by any given domestic organization member or authorized group members.

Some embodiments of the present invention include the following features, characteristics and/or advantages: (i) having the convenience of separating the medical prescriptions, and medical reports, confidential documents of the assets, insurance related documents, bills, etc., all in one place; (ii) no fear of misplacement or losing the physical copies of important documents; (iii) not requiring to separate and organise these documents in the order of importance; (iv) contains sufficient information to refer to whenever and wherever needed; (v) comfort of drawing an analysis of the expenditure in the case of bills accounted for during a time frame; (vi) convenience of record of dates of visit to Doctor/Dentist, and alerting the next probable appointment or the convenience of tracking the insurance payments and the next due date alerts; (vii) getting a dashboard with the information on the health and wealth customised; (viii) providing insights on health representation, the expense flow, and lifestyle or food habits; (ix) refers to medical reports (thereby suggesting insurance recommendations or financial goals and proactive reminders for medical tests); and (x) location wise medical needs based on reports being stored digitally (access given to government authorities, which ultimately allows more and more citizens to start using it and generating more insights).

Embodiments of the present invention provide a document management system that stores all reports, bill, receipts, prescriptions, loans, etc., by converting them into a digital format and separates them using a digital content analysis module.

In one embodiment, the system derives insights from monthly expense bills and or digital payments information and tag them to expenses section.

In one embodiment, the system identifies insurance specific information that is already being paid by a user and compares them against the health records of the organization members by considering medical bills and prescription documents available in the centralized storage system.

Embodiments of the present invention allow users to feed all records of organization members based on their preference. In one embodiment, the system provides recommendation(s) to user(s) on what type of additional insurance or health exercise practices related to existing or predicted health challenges. In another embodiment, the system provides recommendation(s) about financial investment requirements after considering expenses derived in one receipt and also by analyzing spending patterns by a set of users.

In one embodiment, the system correlates lifestyle, eating habits, or purchase patterns based on associated grocery bills against medical reports and suggests required changes to the user's health habits. In another embodiment, when a member visits a doctor for a health check-up, the member's medical history summary will be derived and presented to the doctor from historical reports stored in the centralized system.

Embodiments of the present invention provide comparative insights on the health conditions of the user or other organization member by considering the information fed by the user or by referring the digital documents information provided or uploaded to the system using a digital analysis module.

In some embodiments, details can be added for domestic organization members or an associate group or any community, thereby syncing all the medical needs and goals of the member, as well as properly suggesting budgeting options for the member. In one embodiment, the system proactively suggests medical test requirements based on user financial transactions and available medical records in the DigiDoc system.

In some embodiments, the system reminds users to carry required medication or possible alternates after considering factors including weather, travel history, and/or location attributes. In one embodiment, the system reminds a user if there are no periodic medical tests or medication details registered in their financial transaction history or medical report documents that are uploaded.

Embodiments of the present invention allow medical organizations or government bodies to access the information including planning medical supplies or providing clinical facilities with medical experts to optimally manage and support health needs in a timely and proactive manner.

Embodiments of the present invention proactively suggest or predict possible impacts, necessary precautions or back-up options needed to provide medical support during emergency or critical situations like pandemics by considering medication details of specific users or locality or for that region.

Embodiments of the present invention also learn about changing patterns in health based on advised therapy, food habit changes, medicines, etc. In some cases, the monitored updates are also fed to the system, which eventually can be monetized as benefit to the user in terms of insurance benefits, change of plan, gym discounts, earning health digital badges, etc.

Embodiments of the present invention use this data and can be made available to larger health organizations that helps to identify health related patterns, which can further help in deriving any prospect pandemic situation or alerting nations based on insights derived at the global level.

Embodiments of the present invention leverage the blockchain system for at least the following purposes: (i) to record each transaction as any information received from users, vendors, hospitals, doctors, dietitians, insurance agents, etc., to make sure data is recorded into trusted ledger and is tamper proof; (ii) utilize the blockchain information to generate insights in various aspects and make those insights available for a users individual/group (family) health in a manner that can be presented as a helpful visual graphic; and (iii) uses existing blockchain database information and creates a new blockchain to store insights only and does not keep any reference personal information (PI) and/or sensitive personal information (SPI) (this data can further be utilized by vendor companies to build insurance plans, discounts, diet changes, gym recommendations, etc.).

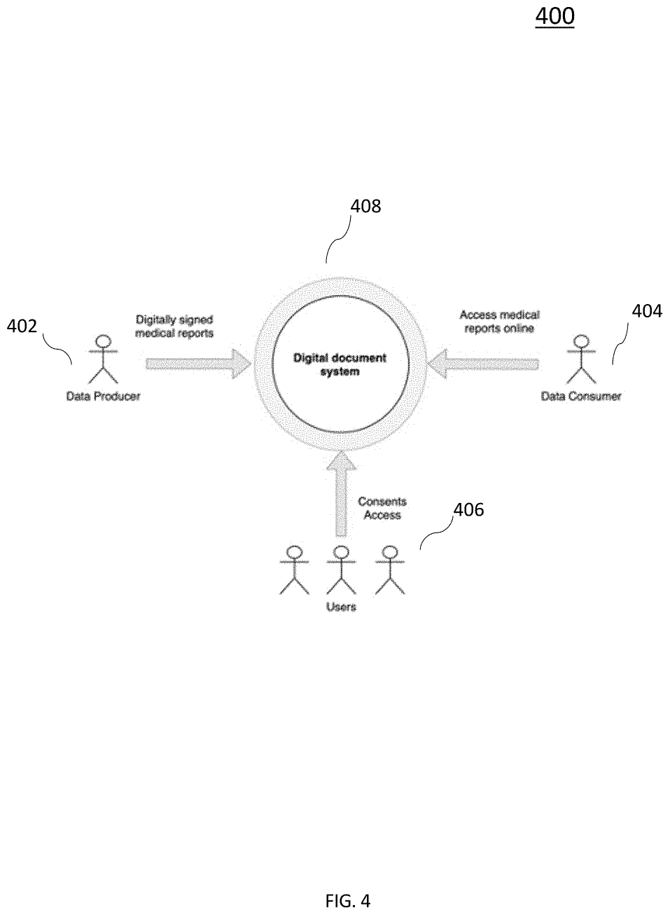

Diagram 400 of FIG. 4 shows an example of how the digital document system is utilized. Diagram 400 includes: data producer 402, data consumer 404, users 406, and digital document system 408.

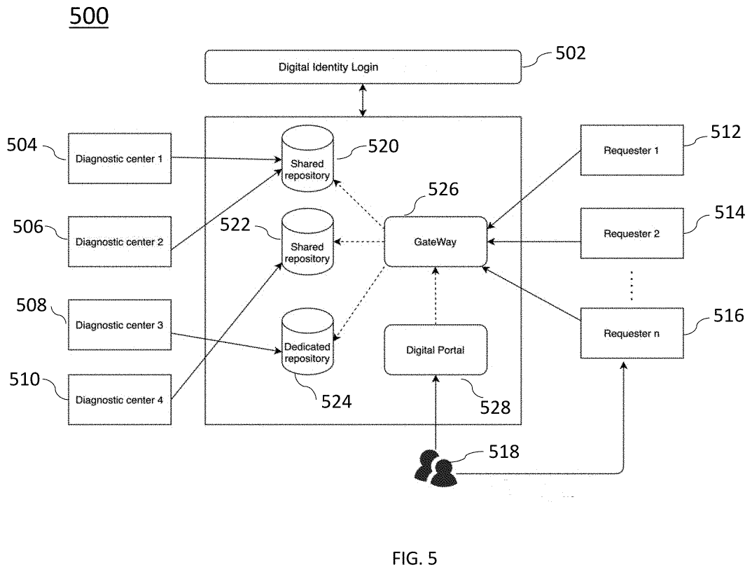

Diagram 500 of FIG. 5 shows a proposed architecture diagram for implementing certain operations of the digital document system. Diagram 500 includes: digital identity login 502, diagnostic center 504, diagnostic center 506, diagnostic center 508, diagnostic center 510, requester 512, requester 514, requester 516, users 518, shared repository 520, shared repository 522, dedicated repository 524, gateway 526, and digital portal 528.

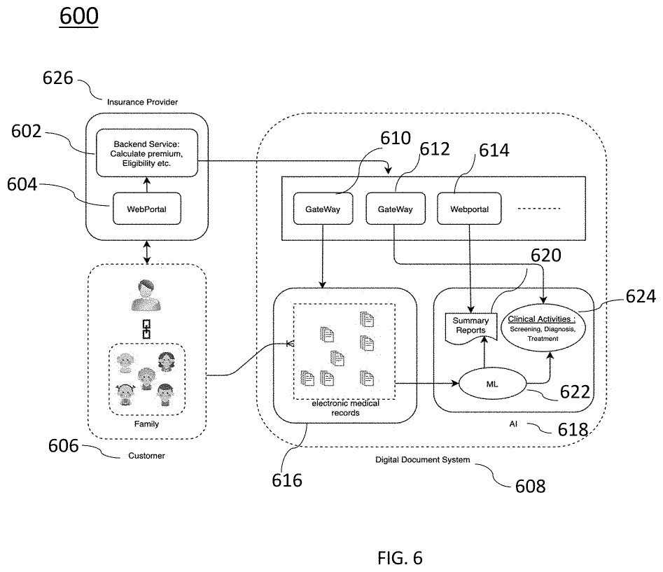

Diagram 600 of FIG. 6 shows a second architecture diagram for implementing embodiments of the present invention. Diagram 600 includes: backend service 602, web portal 604, customer group 606, digital document system 608, gateway 610, gateway 612, web portal 614, electronic records 616, artificial intelligence (AI) module 618, summary reports 620, machine learning (ML) module 622, clinical activities log 624, and insurance provider 626.

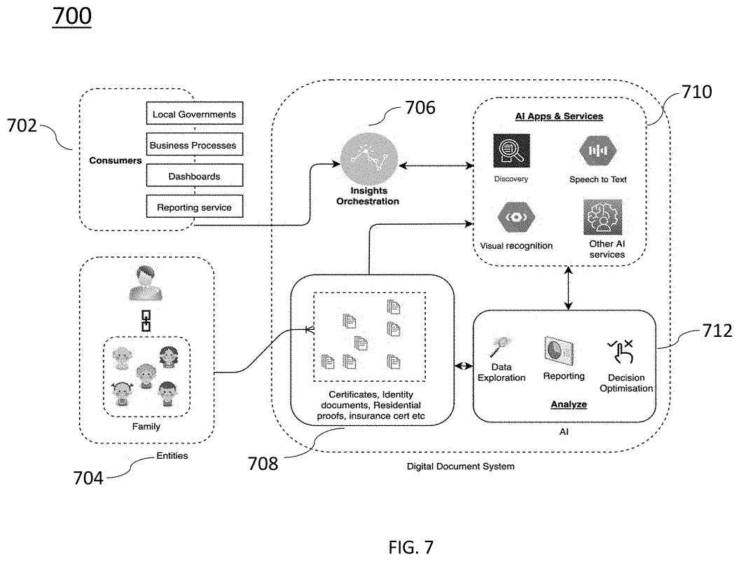

Diagram 700 of FIG. 7 shows a system overview diagram for implementing embodiments of the present invention. Diagram 700 includes: consumers group 702, entities 704, insights orchestration module 706, documents repository 708, AI Apps and Services 710, and AI module 712.

Some embodiments of the present invention use a digital analysis module that applies a document separation algorithm based on file content and text content of the files provided by a user. Some embodiments also use Natural Language Processing (NLP) to capture the context based on for separation with respect to the following: (i) classification (type of content—medical bills, reports or loan documents); (ii) topic/summary (what the substantive content entails); (iii) keywords/phrases/entities (key data points); and (iv) and intent/purpose/request (action being requested).

In some embodiments of the present invention, an insights orchestration module in the system is a digital content analysis module that connects with various Application Programming Interface (API) services such as “Discovery”, “SpeechToText” modules. Other Artificial Intelligence (AI) techniques that are leveraged are “Data Exploration, “Reporting”, “Decision Optimization” to scan and review all available documents such as Certificates, Identity documents, Residential proofs, insurance certificates, loan and expenses receipts, bills and so on.

In some embodiments, an integrated information in digital analysis module will be used to derive insights and help organizations or communities to understand and recommend insurance needs for that individual users and also medical or clinical needs required for that region or community proactively.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

Including/include/includes: unless otherwise explicitly noted, means “including but not necessarily limited to.”

User/subscriber: includes, but is not necessarily limited to, the following: (i) a single individual human; (ii) an artificial intelligence entity with sufficient intelligence to act as a user or subscriber; and/or (iii) a group of related users or subscribers.

Data communication: any sort of data communication scheme now known or to be developed in the future, including wireless communication, wired communication and communication routes that have wireless and wired portions; data communication is not necessarily limited to: (i) direct data communication; (ii) indirect data communication; and/or (iii) data communication where the format, packetization status, medium, encryption status and/or protocol remains constant over the entire course of the data communication.

Receive/provide/send/input/output/report: unless otherwise explicitly specified, these words should not be taken to imply: (i) any particular degree of directness with respect to the relationship between their objects and subjects; and/or (ii) absence of intermediate components, actions and/or things interposed between their objects and subjects.

Without substantial human intervention: a process that occurs automatically (often by operation of machine logic, such as software) with little or no human input; some examples that involve “no substantial human intervention” include: (i) computer is performing complex processing and a human switches the computer to an alternative power supply due to an outage of grid power so that processing continues uninterrupted; (ii) computer is about to perform resource intensive processing, and human confirms that the resource-intensive processing should indeed be undertaken (in this case, the process of confirmation, considered in isolation, is with substantial human intervention, but the resource intensive processing does not include any substantial human intervention, notwithstanding the simple yes-no style confirmation required to be made by a human); and (iii) using machine logic, a computer has made a weighty decision (for example, a decision to ground all airplanes in anticipation of bad weather), but, before implementing the weighty decision the computer must obtain simple yes-no style confirmation from a human source.

Automatically: without any human intervention.

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices

What is claimed is:

1. A computer-implemented method (CIM) comprising:
receiving a plurality of medical documents, with the plurality of medical documents including information indicative of a complete medical history of a user;
responsive to the receipt of the plurality of medical documents, organizing information included in the complete medical history by category, with the categories including treatment(s) received, past ailment(s), and present ailment(s);
creating a digital document repository to store the complete medical history, with the digital repository being accessible by the user and a set of authorized medical professionals; and
storing the complete medical history in the digital document repository.

2. The CIM of claim 1 further comprising:
responsive to the storing of the complete medical history in the digital document repository, using the past ailment(s) information to make a first prediction on a future medical state of the user.

3. The CIM of claim 2 wherein the first prediction on the future medical state is determined, at least in part, by an artificial intelligence (AI) module, with the AI module using at least a portion of the complete medical history as training data.

4. The CIM of claim 1 further comprising:
responsive to the storing of the complete medical history in the digital document repository, using the past ailment(s) information to make a diagnosis on a current medical state of the user.

5. The CIM of claim 1 further comprising:
accessing, by an authorized insurance authority, the digital document repository; and responsive to accessing the digital document repository, using, by the authorized insurance authority, the complete medical history to recommend adjustments to a current insurance policy of the user.

6. The CIM of claim 1 further comprising:
deriving insights into overall medical expenses of a user based on the complete medical history.

7. A computer program product (CPP) comprising:
a machine readable storage device; and
computer code stored on the machine readable storage device, with the computer code including instructions and data for causing a processor(s) set to perform operations including the following:
receiving a plurality of medical documents, with the plurality of medical documents including information indicative of a complete medical history of a user,
responsive to the receipt of the plurality of medical documents, organizing information included in the complete medical history by category, with the categories including treatment(s) received, past ailment(s), and present ailment(s),
creating a digital document repository to store the complete medical history, with the digital repository being accessible by the user and a set of authorized medical professionals, and
storing the patient's complete medical history in the digital document repository.

8. The CPP of claim 7 further comprising:
responsive to the storing of the complete medical history in the digital document repository, using the past ailment(s) information to make a first prediction on a future medical state of the user.

9. The CPP of claim 8 wherein the first prediction on the future medical state is determined, at least in part, by an artificial intelligence (AI) module, with the AI module using at least a portion of the complete medical history as training data.

10. The CPP of claim 7 further comprising:
responsive to the storing of the complete medical history in the digital document repository, using the past ailment(s) information to make a diagnosis on a current medical state of the user.

11. The CPP of claim 7 further comprising:
accessing, by an authorized insurance authority, the digital document repository; and
responsive to accessing the digital document repository, using, by the authorized insurance authority, the complete medical history to recommend adjustments to a current insurance policy of the user.

12. The CPP of claim 7 further comprising:
deriving insights into overall medical expenses of a user based on the complete medical history.

13. A computer system (CS) comprising:
a processor(s) set;
a machine readable storage device; and
computer code stored on the machine readable storage device, with the computer code including instructions and data for causing the processor(s) set to perform operations including the following:
receiving a plurality of medical documents, with the plurality of medical documents including information indicative of a complete medical history of a user,
responsive to the receipt of the plurality of medical documents, organizing information included in the complete medical history by category, with the categories including treatment(s) received, past ailment(s), and present ailment(s),
creating a digital document repository to store the complete medical history, with the digital repository being accessible by the user and a set of authorized medical professionals, and
storing the complete medical history in the digital document repository.

14. The CS of claim 13 further comprising:
responsive to the storing of the complete medical history in the digital document repository, using the past ailment(s) information to make a first prediction on a future medical state of the user.

15. The CS of claim 14 wherein the first prediction on the future medical state is determined, at least in part, by an artificial intelligence (AI) module, with the AI module using at least a portion of the complete medical history as training data.

16. The CS of claim 13 further comprising:
responsive to the storing of the complete medical history in the digital document repository, using the past ailment(s) information to make a diagnosis on a current medical state of the user.

17. The CS of claim 13 further comprising:
accessing, by an authorized insurance authority, the digital document repository; and
responsive to accessing the digital document repository, using, by the authorized insurance authority, the complete medical history to recommend adjustments to a current insurance policy of the user.

18. The CS of claim 13 further comprising:
deriving insights into the overall medical expenses of a user based on the complete medical history.
